(19) United States
(12) Patent Application Publication
(10) Pub. No.: US 2013/0072912 A1
(43) Pub. Date: Mar. 21, 2013

(54) RASPING CANNULA

(71) Applicant: Daniel A. Del Vecchio, Wrentham, MA (US)

(72) Inventor: Daniel A. Del Vecchio, Wrentham, MA (US)

(21) Appl. No.: 13/622,714

(22) Filed: Sep. 19, 2012

Related U.S. Application Data
(60) Provisional application No. 61/536,174, filed on Sep. 19, 2011.

(51) Int. Cl. A61M 39/00

(52) U.S. Cl. CPC A61M 39/00
USPC 604/542

(57) ABSTRACT

A rasping cannula uses a variety of cutting surfaces to augment fat and tissue extraction in cosmetic surgical procedures. In one aspect, a cannula may have a number of holes with curved cutting edges. In another aspect, the tip of a cannula may be curved. In this manner, lateral movement of the cannula excises tissue such as fat for faster, more effective removal.

### RELATED APPLICATIONS

This application claims the benefit of U.S. App. No. 61/536,174 filed on Sep. 19, 2011, the entire content of which is hereby incorporated by reference.

### BACKGROUND

Cannulas are surgical instruments, generally formed as tubes, used for delivery or removal of fluid and the like from a body. In cosmetic surgery, a cannula can be used to harvest fat or other tissue, and/or to insert bulk fillers. A cannula with holes for fat harvesting has been proposed, as described for example in U.S. Pat. No. 5,817,050, the entire content of which is hereby incorporated by reference.

Harvesting cannulas of the prior art focus on vacuum forces. There remains a need for improved harvesting cannulas.

### SUMMARY

A rasping cannula uses a variety of cutting surfaces to augment fat and tissue extraction in cosmetic surgical procedures. In one aspect, a cannula may have a number of holes with curved cutting edges. In another aspect, the tip of a cannula may be curved. In this manner, lateral movement of the cannula excises tissue such as fat for faster, more effective removal.

### BRIEF DESCRIPTION OF THE FIGURES

The invention and the following detailed description of certain embodiments thereof may be understood by reference to the following figures:

FIG. 1 shows a rasping cannula.

FIG. 2 shows a rasping cannula.

FIG. 3 shows a side view of a curved edge for a slit of a cannula.

FIG. 4 shows a side view of a curved tip cutting edge for an insertion tip of a cannula.

### DETAILED DESCRIPTION

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus the term “or” should generally be understood to mean “and/or” and so forth.

Disclosed herein are cannulas for use in cosmetic surgical procedures such as liposuction or other fat harvesting. However it will be understood that the invention disclosed herein is not so limited, and the inventive concept may be adapted to other tissue harvesting and/or extraction procedures.

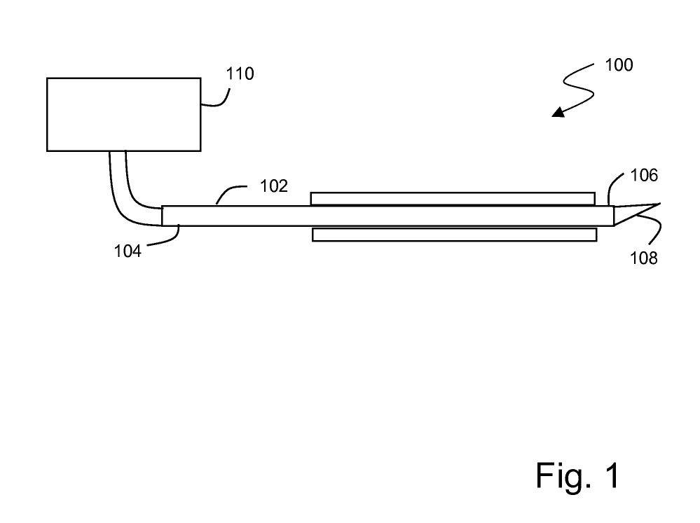

FIG. 1 shows a rasping cannula. The cannula 100 may include a body 102, a proximal end 104, and a distal end 106 with an insertion tip 108.

The proximal end 104 may include any suitable structure for manipulating the cannula 100 or coupling the cannula 100 to a trocar or other instrument. The proximal end 104 may also be coupled by a tube or the like to a vacuum source 110 in fluid communication with an interior of the body 102 for applying vacuum through the body 102 to harvest fat or other tissue through the cannula 100.

The distal end 106 may include an insertion tip 108, which may include an opening and a cutting edge to cut fat or other tissue as the body 102 is inserted into a surgical site.

The cannula 100 may also include a protective sheath 112 configured to slide along the body 102 or otherwise positionable to cover various cutting edges of the cannula 100 in order to controllably expose cutting edges of one or more slits and/or the insertion tip 108. In this manner, the rasping action of the cannula 100 may be selectively deployed at a time and in a location along the body 102 where cutting of fat or other tissue is desired by a user.

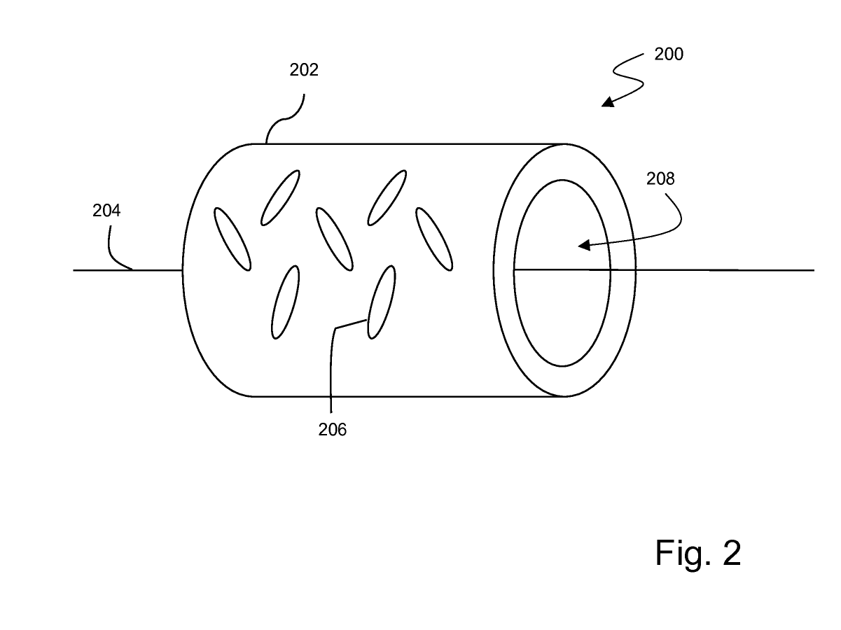

FIG. 2 shows a body 202 of a cannula 200 having an axis 204. By way of example, the body 202 may have an outside diameter of 5.0 mm, 3.5 mm or any other suitable dimensions, and a length of 35 cm or any other length suitable for harvesting fat from a particular site. The body 202 may include an open interior 208 for passage of fat, fluids, other tissue and the like through the body 202 to and from a surgical site. Consistent with conventional surgical implements, the cannula 200 may have a substantially cylindrical body 202, although other cross-sectional shapes may also or instead be used for the body 202, and the cannula 200 may more generally include any elongated body or the like.

A plurality of slits 206 may be provided so that tissue such as fat can be vacuum-extracted along the length of the body 202. Each slit 206 may provide a passage from an exterior of the body 202 to the open interior 208, along with a cutting edge that extends on a perimeter thereof that is elevated outside the body 202 (e.g., away from the open interior 208 and toward the exterior) in order to present a cutting surface that can be used to cut tissue with a corresponding movement of the body 202. In general, a variety of arrangements, shapes, and sizes of slits may be provided, consistent with use in a forward cutting motion of the cannula 200 (i.e., toward the insertion tip), a backward cutting motion (i.e., toward the proximal end), and/or a rotational cutting motion (i.e. rotationally about the axis 204). The cutting edges of the slits 206 may thus be generally oriented to cut tissue in a single direction of motion of the body 202, or in multiple directions.

In one aspect, the cutting edge of each one of the plurality of slits 206 may be oriented to cut tissue in one direction of movement along the axis 204 of the body 202, such as a movement along the axis 204 from the proximal end toward the insertion tip (forward motion) or a movement from the insertion tip toward the proximal end (backward motion) or a bidirectionally upon movement in either direction along the axis (e.g. back and forth, or away from and toward the insertion tip). In another aspect, the cutting edge of each one of the plurality of slits 206 may be oriented to cut tissue upon rotation of the body 202 about the axis 204, which may similarly be (rotationally) one directional or two directional.

A variety of sizes may be used. For example, each slit 206 may be about 1.5 mm along a short (or ‘minor’) axis and about 2.5 mm along a long (or ‘major’) axis, or any similar dimensions for which the major axis is substantially greater than the minor axis. Any number of slits may be provided.

A variety of arrangements may also be used. In one aspect, the slits 206 may wrap substantially completely around a circumference of the body 202. In another aspect, the slits 206 may wrap around about two hundred forty degrees of the body 202, or some other amount that provides a non-harvesting surface so that the cannula 200 can be used as a one-side or directional harvesting device. Similarly, the slits 206 may be oriented with a major axis oriented or angled about forty five degrees off the axis 204 of the body. Some slits may have a major axis oriented or angled about ninety degrees off the major axis of some other ones of the slits, as generally illustrated in FIG. 2, which provides multiple cutting angles and edges as the body 202 moves axially within a surgical site. This arrangement advantageously provides two opposing cutting forces as slits 206 pass over tissue and cut alternately from a left side and a right side.

The shapes of the slits 206 may also vary. For example, while generally oval slits are illustrated, the slits 206 may be circles, polygons, or other shapes, the emphasis being on the direction and arrangement of cutting surfaces presented to tissue as the body moves 202 within tissue rather than the shape of the passage formed into the interior 208 of the body 202. Thus it will be more generally understood that while a specific arrangement of slits is illustrated, different numbers, shapes, and orientations of slits, holes, or the like with cutting edges may also or instead be used with satisfactory results.

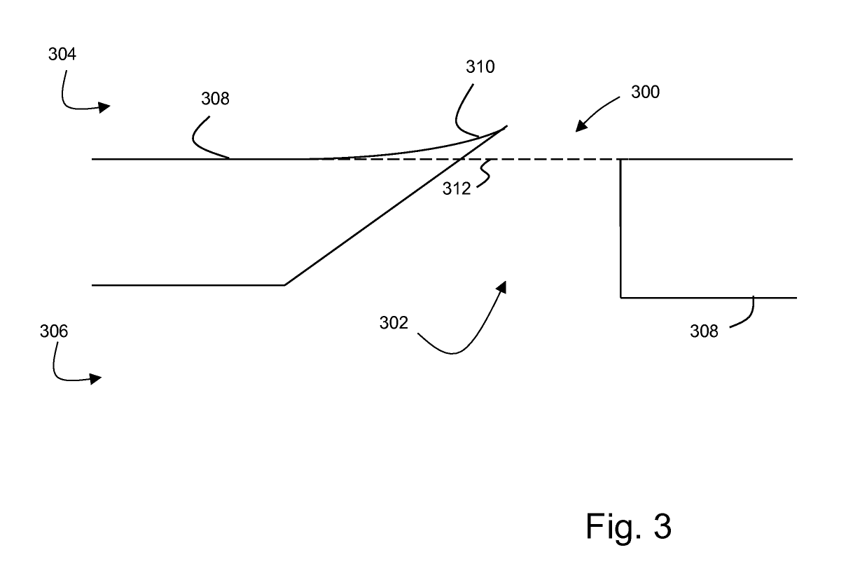

FIG. 3 shows a side view of a curved edge for a slit of a cannula. In general a slit 300 provides an opening 302 between an exterior 304 and an interior 306 of a body 308 of the cannula.

A cutting edge 310 for the slit 300 may be a curved cutting edge forming a sharp, protruding ridge above the surface of the body 308 (the surface of the body illustrated where it spans the slit 300 by a dashed line 312) so that the cutting edge stands up somewhat off the otherwise cylindrical exterior surface of the body 308. This configuration is conveniently machinable to a sharp edge in stainless steel or other biocompatible surgical materials; however it will be understood that any other shape of cutting edge that extends above the surface of the body 308 may also or instead be used. Similarly, the cutting edge 310 may lie in the plane of the surface, which generally provides a less aggressive cutting action that may be advantages in certain applications.

In general, tissue excised from a surgical site by the cutting edge 310 may be further urged toward the interior 306 of the body 308 by the beveled interior surfaces of the slit 300 which, in conjunction with reverse pressure from a vacuum source, can effectively withdraw harvested fat from the site and out of the cannula.

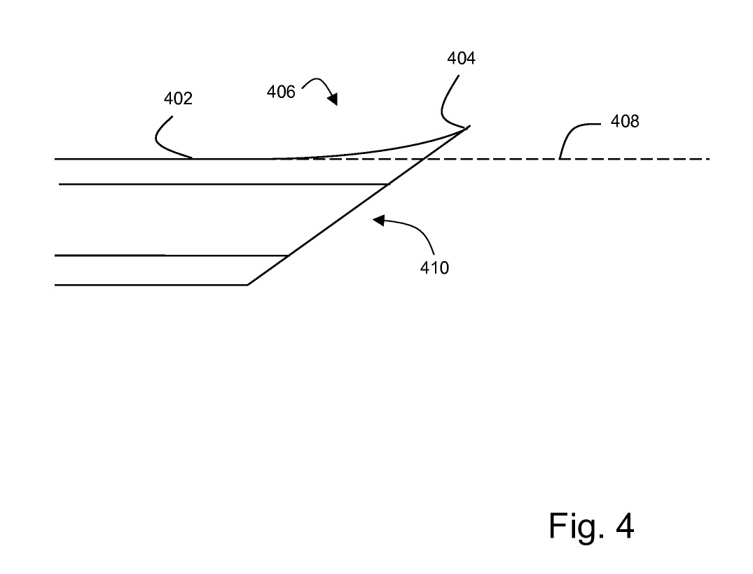

FIG. 4 shows a side view of a curved tip cutting edge for an insertion tip of a cannula. The body 402 of the cannula may have a generally cylindrical exterior with a circular cross section or have some other general cross section along its length. As shown in FIG. 4, the cutting edge 404 of an insertion tip 406 may be on the end of the insertion tip 406 (referred to herein as a “tip cutting edge”) and may form a curved cutting edge that extends beyond the surface of the body 404—that is, radially outside a cross section for the body 404, as illustrated by a dashed line 408—to engage surrounding tissue as generally discussed above.

The insertion tip 406 may include an opening 410 coupled to an open interior of the body 402 through which fat or other tissue can be withdrawn. The cutting edge 404 may be on a perimeter of the opening 410, and may extend entirely around the perimeter or a portion thereof.

As with the cutting edges of the slits discussed above, different positions and angles for the cutting edge 404 may be employed according to a desired aggressiveness for the cutting action. For example, the cutting edge may actually be recessed such that the leading edge of the cutting edge is aligned with the interior surface of the body 102. Or the leading edge may be machined or otherwise formed into a location between the interior and exterior surface of the body 102. More generally, any configuration providing an edge suitable for cutting tissue may be used for the cutting edge as contemplated herein.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A cannula comprising:
an elongated body having an axis, an open interior, a proximal end, and a distal end with an insertion tip; and
a plurality of slits in the elongated body creating passages from an exterior of the elongated body to the open interior, each one of the plurality of slits having a major axis substantially longer than a minor axis and a cutting edge on a perimeter thereof elevated outside the elongated body.

2. The cannula of claim 1 wherein the cutting edge of each one of the plurality of slits is oriented to cut tissue in one direction of movement along the axis by the elongated body.

3. The cannula of claim 2 wherein the one direction of movement is from the proximal end toward the insertion tip.

4. The cannula of claim 2 wherein the one direction of movement is from the insertion tip toward the proximal end.

5. The cannula of claim 1 wherein the cutting edge of each one of the plurality of slits is oriented to cut tissue upon rotation of the elongated body about the axis.

6. The cannula of claim 1 wherein the cutting edge of each one of the plurality of slits is oriented to cut tissue bidirectionally upon movement along the axis by the elongated body toward or away from the insertion tip.

7. The cannula of claim 1 wherein the cutting edge of each one of the plurality of slits includes a curved edge.

8. The cannula of claim 1 further comprising a protective sheath to controllably expose the cutting edge of one or more of the plurality of slits.

9. The cannula of claim 1 further comprising a vacuum source coupled to the proximal end of the open interior.

10. The cannula of claim 1 wherein one or more of the plurality of slits are positioned with the major axis angled about forty five degrees to the axis of the elongated body.

11. The cannula of claim 1 wherein one of the plurality of slits has a first major axis angled about ninety degrees to a second major axis of a second one of the plurality of slits.

12. The cannula of claim 1 wherein the insertion tip includes an opening coupled to the open interior.

13. The cannula of claim 12 wherein the insertion tip includes a cutting edge.

14. The cannula of claim 13 wherein the cutting edge includes a curved edge.

15. The cannula of claim 1 wherein the elongated body is a substantially cylindrical body having a circular cross section.

16. A cannula comprising:
an elongated body having an axis, an open interior, a proximal end, and a distal end with an insertion tip;
a plurality of slits in the elongated body creating passages from an exterior of the elongated body to the open interior, each one of the plurality of slits having a major axis substantially longer than a minor axis; and
an opening in the insertion tip coupled to the open interior, the opening including a tip cutting edge on a perimeter thereof, wherein the tip cutting edge is a curved edge extending radially outside a cross section of the elongated body.

17. The cannula of claim 16 further comprising a vacuum source coupled to the proximal end.

18. The cannula of claim 16 wherein the elongated body is a substantially cylindrical body having a circular cross section.

19. The cannula of claim 18 wherein the plurality of slits each include a cutting edge extending outside the circular cross section of the substantially cylindrical body.

20. The cannula of claim 19 wherein the cutting edge is a curved edge.
